(19) United States
(12) Patent Application Publication
(10) Pub. No.: US 2003/0103881 A1
(43) Pub. Date: Jun. 5, 2003

(54) System and method for processing and viewing mail

(76) Inventors: Charles A. Lane, Wenonah, NJ (US); David F. O'Neal, Marlton, NJ (US)

Correspondence Address: HALE AND DORR LLP, 300 PARK AVENUE, NEW YORK, NY, 10022, US

(21) Appl. No.: 10/283,749

(22) Filed: Oct. 30, 2002

Related U.S. Application Data
(60) Provisional application No. 60/341,166, filed on Oct. 30, 2001.

(51) Int. Cl. A61L 2/22

(52) U.S. Cl. 422/292; 312/1; 422/68.1

(57) ABSTRACT

The present invention provides an apparatus and method for processing and viewing mail. The apparatus provides a structure for maintaining an environment for processing and viewing mail that is separate from the user so that if the mail is contaminated with biological material the contamination remains separate from the user processing and viewing the mail and contained within the apparatus. The apparatus includes glove box like enclosure for receiving mail, wherein at least one member of the glove box like enclosure is transparent. The apparatus includes gloves attached to glove ports, wherein the gloves allow a user to access and view the mail contained therein while still remaining separate from the environment of the glove box like enclosure.

### CROSS REFERENCE TO RELATED APPLICATION

Priority is herewith claimed under 35 U.S.C. §119(e) from Provisional Patent Application No. 60/341,166, filed Oct. 30, 2001, which is incorporated by reference in its entirety.

### FIELD OF INVENTION

The present invention relates to an apparatus and method for processing and viewing mail and, in particular, relates to an apparatus and method for protecting the recipient of mail and/or individuals involved in the direct routing, sorting, and/or handling of mail from biological contaminants spread through the mail.

### BACKGROUND OF THE INVENTION

Presently, mail is delivered to an individual's mailbox. The mail is then removed by the recipient and brought into their home or work place. The mail is then opened. If the mail is contaminated with biological hazardous material the act of opening the mail exposing the person opening the mail and all those around that person to the contaminants enclosed therein.

In an attempt to limit and/or avoid exposure to biological hazardous material that may be contained in the mail many people use surgical gloves in combination with surgical masks when opening mail. Although, the use of gloves and surgical masks may protect an individual they do nothing to prevent or limit the spread of contaminants throughout the individual's home or office environment.

### SUMMARY OF THE INVENTION

The present invention provides an apparatus and method for protecting the recipient of mail and/or individuals involved in the direct routing, sorting, and/or handling of mail (collectively “the user”) from being contaminated by hazardous materials that may be contained in the mail they open and/or otherwise process. The present invention provides an apparatus and method that allows the recipient of mail and/or individuals involved in the direct routing, sorting, and/or handling of mail to open and/or visually inspect mail in an environment separate and apart from the user.

In one preferred embodiment of the present invention, a glove box like enclosure is used to receive mail. The glove box like enclosure has at least one transparent panel and/or member to allow the user to visually inspect the mail and its contents. The glove box like enclosure also has at least one glove port traversing at least one member and/or side of the glove box like enclosure, wherein the glove port allows access user access to the content of the glove box like enclosure through the use of a glove attached to the glove port.

According to another embodiment of the present invention enclosure for receiving, viewing and/or inspecting mail is mounted on a post and/or wall to allow mail delivery personal to deposit mail thereby completely protecting the recipient from handling mail prior to its being deposited in the enclosure.

The present invention has many advantages over traditional mailboxes and include: containment of biological materials to a limited and predefined area in the event such materials are present in mail; serving as a transport container in the event hazardous materials are discovered in the mail; and preservation of hazardous materials in the event such materials are discovered, which allows investigators to study the hazardous materials so as to further identify them.

### BRIEF DESCRIPTION OF THE DRAWINGS

FIG. 1 illustrates a front elevation view, from the top member, of an inspection enclosure in accordance with the invention.

FIG. 2 illustrates a right elevation view, from a flag side, of the inspection enclosure showing a left glove port in accordance with the invention

FIG. 3 illustrates a rear elevation view of the inspection enclosure showing rear mounting brackets in accordance with the invention.

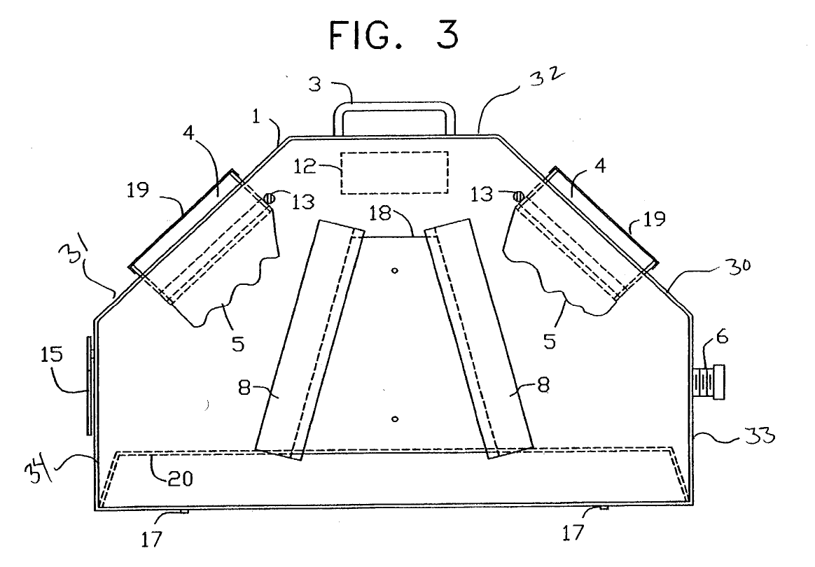

FIG. 4 illustrates a left elevation view of the inspection enclosure showing a right glove port and threaded pipe nipple in accordance with the invention.

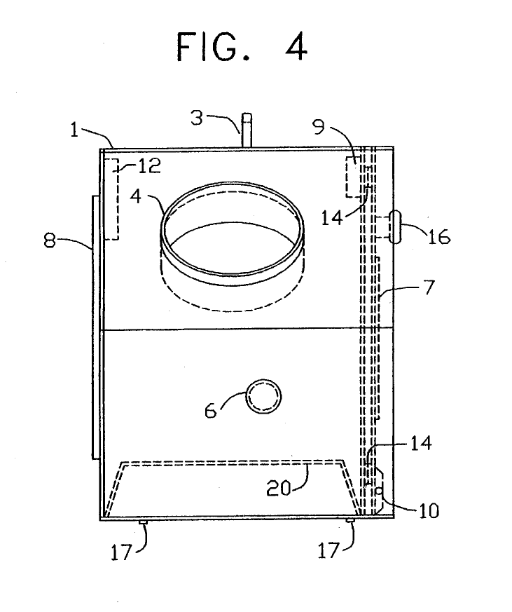

FIG. 5 illustrates a top view of the inspection enclosure showing a lifting handle, an access door handle, glove ports, and rear mounting brackets in accordance with the invention.

FIG. 6 illustrates a multi-user inspection enclosure in accordance with the invention.

### DETAILED DESCRIPTION OF THE PREFERRED EMBODIMENTS

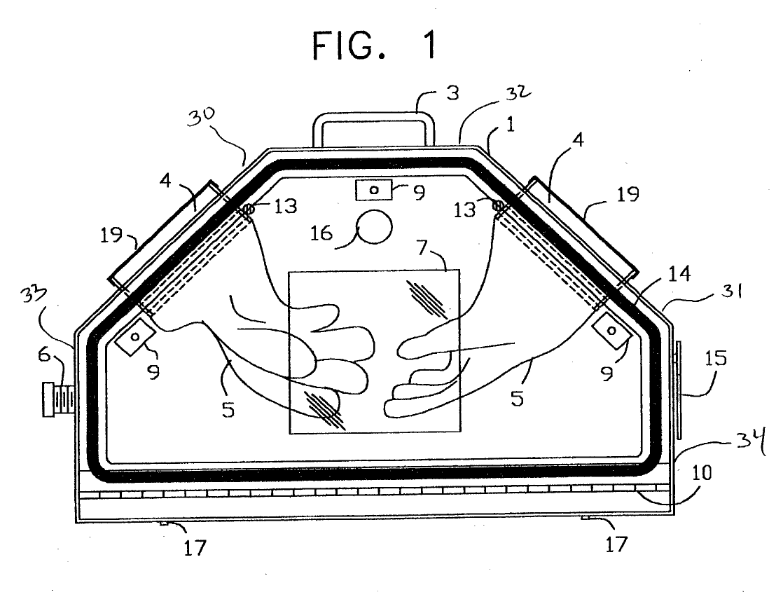

In a preferred embodiment of the present invention, the inspection enclosure is six sided, wherein the six sides are in communication with a top member and a bottom member as illustrated in FIG. 1. The inspection enclosure can be made of, for example, clear plastic and/or other transparent materials like vinyl, latex, rubber, treated paper, Plexiglas, fiberglass, glass or other natural or synthetic materials. In another preferred embodiment of the inspection enclosure not all sides are transparent. In a further embodiment of the present invention, at least one transparent side and/or member is in communication with a magnifying element and/or said at least one transparent side and/or member is a magnifying element. The magnifying element allows the user to visually inspect the mail prior to opening it. In the event the user sees or suspects the presence of a contaminant, such as anthrax, the user can call the local authorities.

Referring to FIG. 1, sides 30 and 31 form four 135-degree interior angles with side 32 and sides 33 and 34 respectively. Traversing sides 30 and 31 are glove port 4. The glove ports 4 allow access to the interior of the inspection enclosure through the use of a glove 5 attached to a glove port 4. The arrangement of sides 30 and 31 relative to the other sides in FIG. 1 allows for natural hand positing when inspecting and/or opening mail. The glove ports 4 can be made of, for example, clear plastic and/or other transparent materials like vinyl, latex, rubber, treated paper, Plexiglas, fiberglass, glass or other natural or synthetic materials. The glove ports 4 can assume any shape that allows a user to place their hand into the interior of the inspection enclosure through a glove 5 attached to the glove port 4. For example, the glove ports 4 can be circular and mounted in sides 30 and 31. According to an embodiment of the present invention, the glove port 4 extends out from the outside surface and the internal surface of at least one side to form a lip. The glove can be secured to either the interior lip or the external lip with a hose type of clamp 13 or other securing mechanism.

The gloves 5 can be made of, for example, clear plastic and/or other transparent materials like vinyl, latex, rubber, treated paper, Plexiglas, fiberglass, glass or other natural or synthetic materials. The inspection enclosure can receive gloves 5 of different sizes. The gloves 5 are removable and replaceable by the user. According to one embodiment of the present invention, each glove 5 allows the user to access the area from a glove port 5 at which the glove is attached to the mid point of the inspection enclosure and sufficiently flexible to handle mail from the smallest to largest type.

The inspection enclosure can be any size. The size of the inspection enclosure can be customized to handle a number of different commercial and residential applications and quantities of mail received.

According to another embodiment of the present invention at least one side of inspection enclosure includes an opening, injection port and/or threaded pipe nipple 6. The injection port 6 allows for the attachment of spray bottle disinfectants and/or other biological neutralizing expectorants. When not in use the injection port 6 is capped.

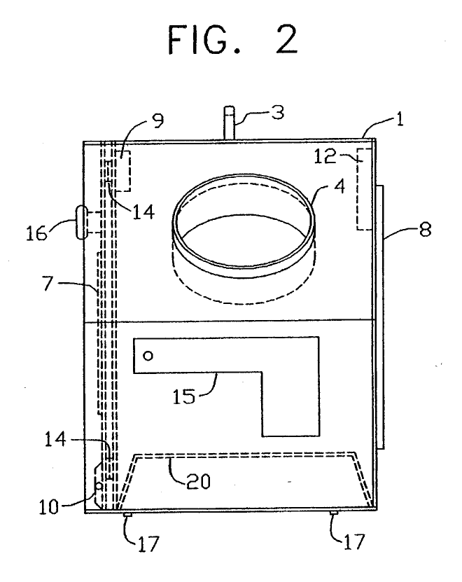

According to an embodiment of the present invention a mounting strip 12, as shown in FIG. 2, for mounting a detector is placed in the interior of the inspection enclosure. The detector indicates the presence of biological material in the mail contained in the inspection enclosure.

According to an embodiment of the present invention, the top member is attached to at least one of said six sides by a hinge 10 and/or other mechanism that allows the top member to be moved relative to at least one of the sides and allow the user access to the interior of the inspection enclosure. The hinge 10 allows the top member to remain attached to at least one of the sides while the top member is open allowing access into the interior of the inspection enclosure. The hinge 10 can be a molded one-piece or a separate piece bonded to the top member and at least one of the sides. The present invention is not limited to a hinge 10 mechanism and includes any mechanism for allowing the top member to remain attached to at least one of the sides when places in a position that permits user access to the interior of the inspection enclosure.

The top member opens upward and away from the user and seals tight when closed. The top member can be made of, for example, clear plastic and/or other transparent materials like vinyl, latex, rubber, treated paper, Plexiglas, fiberglass, glass or other natural or synthetic materials. According to this embodiment, the top member can also include at least one of a gasket material 14 within the lid's perimeter sealing groove, a handle 16 for lifting the entire mailbox and/or the top member, a latch, or a magnifying element. Moreover, each of the above elements can be molded as part of the top member or attached separately. According to another embodiment of the present invention, the top member includes multiple sub-members that open either independently of each other or in combination with each other to allow access to the interior of the inspection enclosure.

According to an embodiment of the present invention, the top member is immovably attached to each side. According to this embodiment, the top member can also include at least one of a handle 16 for lifting the entire mailbox or a magnifying element 7. Moreover, each of the above elements can be molded as part of the top member or attached separately.

According to an embodiment of the present invention, the inspection enclosure includes a latch mechanism 9. The latch mechanism 9 holds the top member in a closed position until the user requires access to the interior of the inspection enclosure and manually releases the latch mechanism 9.

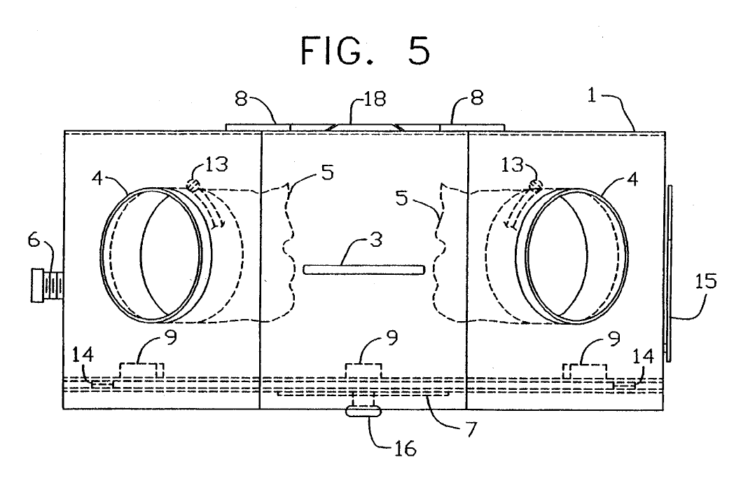

According to another embodiment of the present invention, a mounting assembly 8 is in communication with the bottom member, wherein the mounting assembly 8 is used for securing the inspection enclosure to a mailbox pole and/or wall. The mounting assembly 8 allows the inspection enclosure to be easily slid and/or attached/detached to a mounting plate 18, as shown in FIG. 5, on a mailbox pole or wall yet robust enough to withstand the elements of nature. The assembly 8, 18, in combination, can be, for example, a sliding type, mounting snap clips or a speed type wing nut mounting.

According to another embodiment of the present invention, a plurality of rubber feet 17 are attached to at least one of the sides. The rubber feet 17 act to avoid scratching surfaces the inspection enclosure may be place on.

According to another embodiment of the present invention, the inspection enclosure encloses a condensation tray 20, as shown in FIG. 2. The condensation tray 20 acts to keep received mail from setting in water when the inspection enclosure of the present invention is used for curbside mail service. The surface condensation tray 20 can be dimpled or corrugated or possess a sinusoidal shape or other shape that results in lands and pits and/or peaks and valleys.

According to an embodiment of the present invention, a flag is adjustably attached to at least one of the sides. The position of the flag acts a signal to the mail carrier as to the presence or absence of mail in the inspection enclosure.

The inspection enclosure can be manufactured using a mold type process forming a one piece design including but not limited to the lid, portals/hubs, threaded nipples, magnifying viewing window, mounting strip, hinge, latch, and bottom mounting bracket. It may also be comprised of individual pieces bonded together.

The apparatus and method of the present invention can be implemented in a number of ways, an example of which follows. The inspection enclosure is collected from its post and/or wall where it receives mail. The user slides the unit off its mount and takes it inside their home or office, laying it on its bottom member. The user then removes the two glove port caps 19 and inserts their hands into gauntlet gloves, picks up a letter and begins opening and inspecting its contents. In the event the user becomes suspicious about their mail or its contents they simply remove their hands from the inspection enclosure and call the proper authorities, i.e., 911. In the event the user is satisfied with what they have seen in their mail and believe no biological material is present the user opens the access door (i.e., top member) and removes the mail.

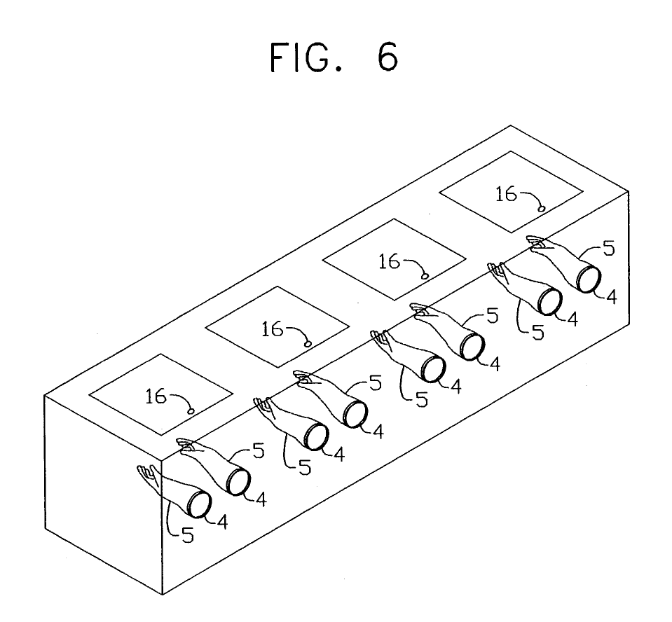

It should be understood that the present invention is not limited to an inspection enclosure with a predefined number of sides but includes inspection enclosures with three sides as well as twenty sides or greater as well as everything between three sides and twenty sides. For example, an inspection enclosure the size of a large conference table with multiple glove ports, as shown in FIG. 6, allows multiple people to sort and inspect mail simultaneously. Additional, the inspection enclosure can be fitted to a photocopy machine and allow users to make photocopies of mail using the photocopy machine, destroying the originals documents and routing the photocopies of the elements of the mail to the intended recipients. Users would be able to open, inspect and photocopy a piece of mail without coming in direct contact with said mail. Further, larger sized inspection enclosures, such as a multiple user construct, can have multiple access openings for the purpose of loading or discarding mail. These access openings can have spring loaded doors to allow mail to flow through a chute to a waste receptacle or even a smaller individual sized inspection enclosure for further distribution, i.e., mail is sorted as to recipient in a large inspection enclosure in a company's mailroom, then is pushed through a chute, into a smaller inspection enclosure for opening and inspection in a specific recipient's office.

It should also be understood that each of the above embodiments described above can be readily combined.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. An apparatus for receiving and processing mail, said apparatus comprising:
a first member coupled to a plurality of side members;
a second member connected to at least one of said plurality of side members by a hinge means and in communication with said plurality of side members;
wherein at least one of said first member, said second member, said plurality of side members, or combinations thereof is transparent;
at least one sight magnifier attached to at least one of said first member, said second member, said plurality of side members, or combinations thereof.
at least one glove port traversing at least one of said plurality of side members; and
a glove having a sleeve portion attached to said at least one glove port.

2. The apparatus according to claim 1, wherein said second member is in communication with said plurality of side members by a gasket means.

3. The apparatus according to claim 1, wherein said first member and said plurality of side members form an integrated structure.

4. The apparatus according to claim 3, wherein said integrated structure is formed by injection molding.

5. The apparatus according to claim 1, wherein said second member is parallel to said first member.

6. The apparatus according to claim 1, wherein said first member and said second member are perpendicular to said plurality of side members.

7. The apparatus according to claim 1, wherein said at least one of said plurality of side members attached to said glove port comprises a first surface and a second surface and said glove port comprises an interior lip that extends away from said first surface and an exterior lip that extends away from said second surface.

8. The apparatus according to claim 7, wherein said sleeve portion of said glove is attached to said interior lip by a clamping means.

9. The apparatus according to claim 7, further comprising an access cap attached to said exterior lip.

10. The apparatus according to claim 1, wherein at least one of said first member, said second member, said plurality of side members, or combinations thereof comprises at least one of clear plastic, vinyl, latex, rubber, treated paper, Plexiglass, fiberglass, glass, or combinations thereof.

11. The apparatus according to claim 1, further comprising an injection portal in at least one of said first member, said second member, said plurality of side members, or combinations thereof.

12. The apparatus according to claim 11, wherein said injection portal is a threaded pipe nipple.

13. The apparatus according to claim 1, further comprising a mounting plate attached to at least one of said first member, said second member, said plurality of side member, or combinations thereof.

14. The apparatus according to claim 1, further comprising a detector for detecting the presence of biological material.

15. An apparatus for receiving and processing mail, said apparatus comprising:
a first member coupled to a plurality of side members;
a second member coupled to said plurality of side members;
wherein at least one of said first member, said second member, at least one of said plurality of side members, or combinations thereof is transparent;
at least one glove port traversing at least one of said plurality of side members; and
a glove having a sleeve portion attached to said at least one glove port.

16. The apparatus according to claim 15, wherein said first member, said second member, and said plurality of side members form an integrated structure.

17. The apparatus according to claim 16, wherein said integrated structure is formed by an injection molding process.

18. The apparatus according to claim 15, wherein said second member is parallel to said first member.

19. The apparatus according to claim 15, wherein said first member and said second member are perpendicular to said plurality of side members.

20. The apparatus according to claim 15, wherein said at least one of said plurality of side members attached to said glove port comprises a first surface and a second surface and said glove port comprises an interior lip that extends away from said first surface and an exterior lip that extends from said second surface of at least one of said plurality of side members.

21. The apparatus according to claim 20, wherein said sleeve portion of said glove is attached to said interior lip by a clamping means.

22. The apparatus according to claim 20, further comprising an access cap attached to said exterior lip.

23. The apparatus according to claim 15, wherein at least one of said first member, said second member, said plurality of side members, or combinations thereof comprises at least one of clear plastic, vinyl, latex, rubber, treated paper, Plexiglass, fiberglass, glass, or combinations thereof.

24. The apparatus according to claim 15, further comprising an injection port in at least one of said first member, said second member, said plurality of side members, or combinations thereof.

25. The apparatus according to claim 24, wherein said injection portal is a threaded pipe nipple.

26. The apparatus according to claim 15, further comprising a mounting plate attached to at least one of said first member, said second member, said plurality of side member, or combinations thereof.

27. The apparatus according to claim 15, further comprising a detector for detecting the presence of biological material.

28. An apparatus for receiving and processing mail, said apparatus comprising:
a first member coupled to a plurality of side members;
a second member connected to at least one of said plurality of side members by a hinge means and in communication with said plurality of side members by a gasket means,
wherein at least one of said first member, said second member, at least one of said plurality of side members, or combinations thereof is transparent;
at least one sight magnifier attached to at least one of said first member, said second member, at least one of said plurality of side members, or combinations thereof.
a flag adjustably attached to at least one of said plurality of side members, said first member, or said second member;
a first handle attached to at least one of said plurality of side members;
a second handle attached to said second member;
at least one glove port traversing at least one of said plurality of side members; and
a glove having a sleeve portion attached to said at least one glove port.

29. The apparatus according to claim 28, further comprising a mounting plate attached to at least one of said first member, said second member, at least one of said plurality of side members, or combinations thereof.

30. A method for receiving and processing mail comprising:
receiving mail in a glove box, wherein said glove box comprises
a first member coupled to a plurality of side members;
a second member coupled to said plurality of side members;
wherein at least one of said first member, said second member, at least one of said plurality of side members, or combinations thereof is transparent;
at least one glove port traversing at least one of said plurality of side members;
a glove having a sleeve portion attached to said at least one glove port; and
inspecting mail for the presence of biological material.

31. The method according to claim 30, further comprising routing mail to a plurality of portable glove boxes, wherein each of said plurality of portable glove boxes comprises
a first member coupled to a plurality of side members;
a second member connected to at least one of said plurality of side members by a hinge means and in communication with said plurality of side members by a gasket means,
wherein at least one of said first member, said second member, said plurality of side members, or combinations thereof is transparent;
at least one sight magnifier attached to at least one of said first member, said second member, said plurality of side members, or combinations thereof.
at least one glove port traversing at least one of said plurality of side members; and
a glove having a sleeve portion attached to said at least one glove port.

32. A method for receiving and processing mail comprising:
receiving mail in a glove box, wherein said glove box comprises
a first member coupled to a plurality of side members;
a second member coupled to said plurality of side members;
wherein at least one of said first member, said second member, at least one of said plurality of side members, or combinations thereof is transparent;
at least one glove port traversing at least one of said plurality of side members;
a glove having a sleeve portion attached to said at least one glove port;
opening mail inside said glove box;
photocopying inside said glove box each element of opened mail; and
routing a photocopy of each element of opened mail to recipient.

33. The method according to claim 32, further comprising disposing of said opened mail.

34. A method for receiving and processing mail comprising:
providing a glove box for receiving mail, wherein said glove box comprises
a first member coupled to a plurality of side members;
a second member coupled to said plurality of side members;
wherein at least one of said first member, said second member, at least one of said plurality of side members, or combinations thereof is transparent;
at least one glove port traversing at least one of said plurality of side members;
a glove having a sleeve portion attached to said at least one glove port;
an injection port traversing at least one of said first member, said second member, said plurality of side members, or combinations thereof; and
applying an aerosol through said injection port to said glove box contents.
